(19) United States
(12) Patent Application Publication
(10) Pub. No.: US 2010/0189703 A1
(43) Pub. Date: Jul. 29, 2010

(54) NON-DESTRUCTIVE METHOD TO QUANTIFY AND ISOLATE ANTIGEN-SPECIFIC B CELLS AND USES THEREOF

(76) Inventors: George K. Lewis, Baltimore, MD (US); Anthony L. Devico, Alexandria, VA (US); Robin Flinko, Chester, MD (US)

Correspondence Address: MOORE & VAN ALLEN PLLC, P.O. BOX 13706, Research Triangle Park, NC, 27709, US

(21) Appl. No.: 12/593,037

(22) PCT Filed: Mar. 26, 2008
(86) PCT No.: PCT/US08/58180

§ 371 Date: Apr. 1, 2010

Related U.S. Application Data
(60) Provisional application No. 60/908,037, filed on Mar. 26, 2007.

(51) Int. Cl. A61K 35/12; G01N 33/53

(52) U.S. Cl. 424/93.71; 435/7.24

(57) ABSTRACT

The present invention relates to antigen-Ig Ab fusion molecules and methods of using same, wherein antigen-Ig Ab fusion molecules include an antigen fused to an immunoglobulin molecule, fragment or variant thereof and wherein the fusing of the immunoglobulin molecule to the antigen does not alter the specificity or tertiary structure of important epitopes of the antigen. This method allows the direct quantification and isolation of antigen-specific B cells by flow cytometry in essentially any species.

### CROSS-REFERENCE TO RELATED APPLICATION

The present application claims priority to U.S. Provisional Patent Application No. 60/908,037 filed on Mar. 26, 2007, the content of which is hereby incorporated by reference herein.

### BACKGROUND THE INVENTION

1. Field of the Invention

This invention relates generally to isolation of cells having affinity for a specific antigen, and more specifically, to a method of quantifying and isolating antigen-specific B cells for further use including production of monoclonal antibodies and quantifying antigen-specific B cells.

2. Description of Related Art

The discovery by Kohler and Milstein of murine hybridomas capable of secreting specific monoclonal antibodies against predetermined antigens ushered a new era in the field of clinical immunology. The clonal selection and immortality of such hybridoma cell lines assure the monoclonality, monospecificity and permanent availability of their antibodies.

Generation of human monoclonal antibodies has been practically difficult for a number of reasons. First, it is not practical to immunize a human being with an immunogen of interest. Instead, the human antibodies which have been produced have been based on the adventitious presence of an available spleen. While four alternative ways of generating human monoclonal antibodies with desired antigen-binding specificity have been developed, they all suffer from a similar problem, that being isolation of the most appropriate B cell for a specific antigen. Heretofore, the antigen has been modified to isolate the B cells with the greatest affinity, often disrupting the important epitopes on the antigen thereby defeating the purpose for isolating and separating the B cells with the ability to generate the correct antibody.

For example, the selection of B cells immunoreactive with an antigen involves the following steps: (a) contacting a population of human lymphocytes comprising B cells with the desired antigen under conditions favorable for specific binding of B lymphocytes to the desired antigen; and (b) separating the unbound B lymphocytes from the B lymphocytes bound to the desired antigen. This type of separation typically proceeds with layering the antigens onto a solid substrate, followed by plating B cells over the layer of antigens. The B cells are then allowed to bind to the antigens under physiological conditions, wherein the pH is maintained between 6 and 8 and the temperature is between about 200° to 40° C. The unbound B lymphocytes are removed by washing, aspiration or any other suitable means. Notably, the antigens have to be secured to the solid substrate which can cause modification of the tertiary structure of the antigen thus changing the binding profile of the B cells. Clearly, disrupting the epitope of the antigen can defeat the purpose of locating the most promising B cell with the greatest affinity for the correct tertiary structure of the antigen. Further, B cell enrichment is reduced because of interaction of B cells with weak antigenic epitopes.

Thus, there is a need for a method for separating antigen-specific B cells with the use of an antigen that has maintained the integrity of its tertiary structure thereby providing high specificity for antigen-specific B cell enrichment.

### SUMMARY OF THE INVENTION

The present invention relates to a general method for separating antigen-specific B-cells by providing a hinged/fused selectable marker coupled to a non-constrained/restricted/modified antigen of interest; incubating with B-cells that respond to Antigen being used to allow for binding of antigen to B cell and sorting B cell-Ag-linker-marker complex by a non-destructive physical means in order to isolate the complex.

A particularly preferred method relates to a method separating antigen-specific B-cells by modifying an antigen that has a desired reactive epitope with the fusion of at least a fragment of an Ig Ab, variant thereof; a Zn finger protein, a FLAG peptide, a magnetic particle, or an enzyme, and detecting the binding of the antigen-Ig Ab fusion peptide to the antigen-specific B cells.

In one aspect, the present invention relates to fusing the heavy chain of the human IgG1 protein with an antigen for detecting antigen-specific B cells without altering the tertiary structure of the antigenic epitopes.

In yet another aspect, the present invention relates to a modified antigen comprising an antigen fused to at least a fragment of the heavy chain of IgG1 protein and further comprising a fluorescent tag, wherein the fluorescent tag may include a fluorescently tagged Fab fragment specific for the Fc region of human IgG1 or a fusion fluorescent molecule comprising the IgG1 CH3 region and a fluorescent reporter molecule attached thereto.

The present invention relates to a method for separating or isolating antigen-specific B-cells comprising:
a) modifying the antigen with the fusion of at least a fragment of a human IgG1 antibody or variant thereof to generate an antigen-IgG1 fusion peptide, wherein the antigen comprises a reactive epitope;
b) contacting a sample comprising B cell suspected of being previously exposed to the antigen with the antigen-IgG1 fusion peptide; and
c) detecting the binding of the antigen-IgG1 fusion peptide to antigen-specific B-cells by flow cytometry.

In a still further aspect, the present invention relates to a kit comprising a fusion protein comprising an antigen fused to at least a fragment of an IgG antibody or variant thereof and a fluorescent tag connected to the IgG antibody.

In another aspect, the present invention relates to a method of treatment comprising:
a) isolating B-cells from a subject;
b) modifying an antigen with the fusion of at least a fragment of an Ig Ab or variant thereof, wherein the antigen comprises a reactive epitope;
c) contacting the antigen-Ig Ab fusion peptide to the isolated B-cells;
d) detecting the binding of the antigen-Ig Ab fusion peptide to antigen-specific B cells in a biological sample of a subject, wherein the antigen-specific B cells are specific for the reactive epitope;
e) separating the antigen-specific B cells from the antigen-Ig Ab fusion peptide;
f) treating the antigen-specific B cells with a reagent including a cytokine, a chemotherapeutic agent, HIV therapeutic agent, chemokine or an antibody, and
g) reinfusing the treated antigen-specific B cells into the subject to provide a therapeutic effect.

In a still further aspect, the present invention relates to a method for quantifying antigenic specific B cells, the method comprising:
a) modifying an antigen with the fusion of at least a fragment of an Ig Ab or variant thereof, wherein the antigen comprises a reactive epitope;
b) contacting the antigen-Ig Ab fusion peptide to a sample comprising B cells suspected of being previously exposed to the antigen;
c) detecting the binding of the antigen-Ig Ab fusion peptide to antigen-specific B cells in the sample; and
d) quantifying the antigen-specific B cells by flow cytometry.

Another aspect of the present invention relates to a method for measuring the immune response elicited by a vaccine comprising an antigen, the method comprising:
a) administering the vaccine to a mammal, wherein the vaccine comprises an antigen;
b) isolating B-cells from the mammal, wherein the B-cells are suspected of being exposed to the antigen;
c) modifying the antigen with the fusion of at least a fragment of an Ig Ab or variant thereof; contacting the antigen-Ig Ab fusion peptide to the isolated B cells;
d) detecting the binding of the antigen-Ig Ab fusion peptide to antigen-specific B cells in the sample of the mammal;
e) separating the antigenic specific B cells from the antigen-Ig Ab fusion peptide; and
f) quantifying the number of antigen-specific B cells elicited by the vaccine.

A still further aspect of the present invention relates to a method for determining and quantifying the frequencies of B cells for different epitopes on the same antigen, the method comprising:
a) generating at least two different antigen-IgAb fusion peptides with different luminescent label, wherein the different antigen-IgAb have different active epitopes of the antigen;
b) contacting the antigen-Ig Ab fusion peptides to a sample comprising B cells; and
c) detecting the binding of the different antigen-Ig Ab fusion peptides to different antigen-specific B cells in a biological sample of a subject and quantifying same.

In another aspect, the present invention relates to an in vitro method for generating and isolating antigen-specific antibody producing B cell comprising:
a) removing a blood sample from a donor;
b) isolating at least naive T cells, naive B cells and monocytes from the sample;
c) differentiating the monocytes into monocyte derived dendritic cells;
d) culturing the monocytes derived dendritic cells with T cell, B cells and the target antigen to generate antigen-specific antibody producing B cells;
e) modifying the target antigen with the fusion of at least a fragment of an Ig Ab or variant thereof;
f) contacting the antigen-Ig Ab fusion peptide to the antigen-specific antibody producing B cells;
g) detecting the binding of the antigen-Ig Ab fusion peptide to antigen-specific antibody producing B cells; and
h) separating the antigen-specific antibody producing B cells.

Other aspects and advantages of the invention will be more fully apparent from the ensuing disclosure and appended claims

### BRIEF DESCRIPTION OF THE FIGURES

FIG. 1 is a schematic diagram of as antigen-IgG fusion peptide of the present invention.

FIGS. 2A, 2B and 2C show the results of the FLSC-IgG1 fusion peptide when reacted with transfected dog lymphocytes that express CCR5 and analysis by flow cytometry.

FIG. 3 shows the results of FLSC-IgG Zenon to 19e B cells that are FLSC specific.

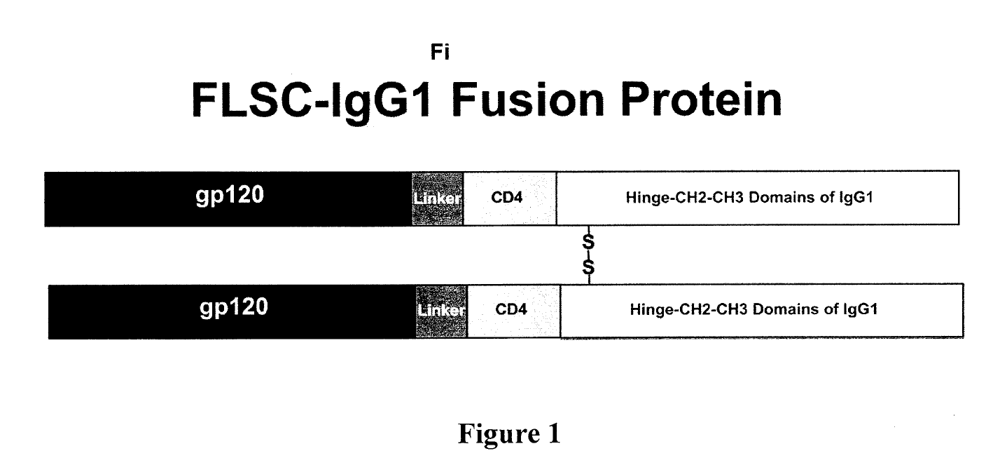

### DETAILED DESCRIPTION OF THE INVENTION

The present invention provides for antigen-Ig Ab fusion molecules and methods of using same, wherein antigen-Ig Ab fusion molecules include an antigen fused to an immunoglobulin molecule, fragment or variant thereof and wherein the fusing of the immunoglobulin molecule to the antigen does not alter the specificity or tertiary structure of important, desired and reactive epitopes of the antigen. This method allows the direct quantification and isolation of antigen-specific B cells by flow cytometry in essentially any species.

These enriched antigen-specific B cells can be used for monoclonal antibody production by any of a variety of species specific methods including: a) primary immunization in vitro with antigen, dendritic cells, and helper T cells followed by EBV transformation to produce stable cell lines (humans and nonhuman primates); b) mitogen activation and fusion with a hybridoma fusion partner (humans, rats, mice, and rabbits); or d) PCR amplification of VH and VL genes from single sorted B cells and cloning into immunoglobulin expression vectors (essentially any vertebrate species that makes conventional antibodies). In addition, this method allows the direct quantification of B cells in any tissue source, which can be an important measure of immune responses in the clinical laboratory and in monitoring immune responses elicited by vaccines.

### DEFINITIONS

The practice of the present invention will employ, unless otherwise indicated, conventional techniques of immunology, molecular biology, microbiology, cell biology and recombinant DNA, which are within the skill of the art. See, e.g., Sambrook, et al. MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

The terms “immunoglobulin molecule” or “antibodies,” as used herein, mean molecules that contain an antigen binding site which specifically binds an antigen. Structurally, the simplest naturally occurring antibody (e.g., IgG) comprises four polypeptide chains, two heavy (H) chains and two light (L) chains inter-connected by disulfide bonds. The natural immunoglobulins represent a large family of molecules that include several types of molecules, such as IgD, IgG, IgA, IgM and IgE. The term also encompasses hybrid antibodies, or altered antibodies, and fragments thereof, including but not limited to Fab fragment(s), and Fv fragment. These fragments are also termed “antigen-binding fragments”. Examples of binding fragments encompassed within the term “antigen-binding fragments” include but are not limited to (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment which consists of a VH domain; (v) an isolated complementarity determining region (CDR); and (vi) an F(ab′)₂ fragment, a bivalent fragment comprising two Fab fragments linked by a disulfide bridge at the hinge region. Furthermore, although the two domains of the Fv fragment are generally coded for by separate genes, a synthetic linker can be made that enables them to be made as a single protein chain (known as single chain Fv (scFv) by recombinant methods. Such single chain antibodies are also encompassed within the term “antigen-binding fragments”. Preferred antibody fragments are those which are capable of crosslinking their target antigen, e.g., bivalent fragments such as F(ab′)₂ fragments. Alternatively, an antibody fragment which does not itself crosslink its target antigen (e.g., a Fab fragment) can be used in conjunction with a secondary antibody which serves to crosslink the antibody fragment, thereby crosslinking the target antigen.

Antibodies can be fragmented using conventional techniques as described herein and the fragments screened for utility in the same manner as described for whole antibodies. A Fab fragment of an immunoglobulin molecule is a multimeric protein consisting of the portion of an immunoglobulin molecule containing the immunologically active portions of an immunoglobulin heavy chain and an immunoglobulin light chain covalently coupled together and capable of specifically combining with an antigen. Fab fragments can be prepared by proteolytic digestion of substantially intact immunoglobulin molecules with papain using methods that are well known in the art. However, a Fab fragment may also be prepared by expressing in a suitable host cell the desired portions of immunoglobulin heavy chain and immunoglobulin light chain using methods disclosed herein or any other methods known in the art.

An Fv fragment of an immunoglobulin molecule is a multimeric protein consisting of the immunologically active portions of an immunoglobulin heavy chain variable region and an immunoglobulin light chain variable region covalently coupled together and capable of specifically combining with an antigen. Fv fragments are typically prepared by expressing in suitable host cell the desired portions of immunoglobulin heavy chain variable region and immunoglobulin light chain variable region using methods described herein and/or other methods known to artisans in the field.

The term “antigen,” as used herein means a substance that is recognized and bound specifically by an antibody. Antigens include but are not limited to peptides, proteins, glycoproteins, polysaccharides and lipids; portions thereof and combinations thereof.

The term “recombinant polynucleotide” as used herein means a polynucleotide of genomic, cDNA, semisynthetic, or synthetic origin which either does not occur in nature or is linked to another polynucleotide in a normatural arrangement.

The terms “operably linked” or “operatively linked,” as used herein means a juxtaposition wherein the components so described are in a relationship permitting them to function in their intended manner.

The term “vector,” as used herein means a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions.

The term “expression vectors,” as used herein means polynucleotides which, when introduced into an appropriate host cell, can be transcribed and translated into a polypeptide(s). An “expression system” usually connotes a suitable host cell comprised of an expression vector that can function to yield a desired expression product.

The term “host cell,” as used herein includes an individual cell or cell culture which can be or has been a recipient for vector(s) or for incorporation of nucleic acid molecules and/or proteins. Host cells include progeny of a single host cell, and the progeny may not necessarily be completely identical (in morphology or in genomic of total DNA complement) to the original parent cell due to natural, accidental, or deliberate mutation. A host cell includes cells transfected in vivo with a polynucleotide(s) of this invention.

The terms “transformation” or “transfection,” as used herein mean the insertion of an exogenous polynucleotide into a host cell, irrespective of the method used for the insertion, for example, lipofection, transduction, infection or electroporation. The exogenous polynucleotide may be maintained as a non-integrated vector, for example, a plasmid, or alternatively, may be integrated into the host cell genome.

The terms “polypeptide”, “peptide” and “protein,” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

Antigens

Antigens useful for the present invention may be a foreign antigen, an endogenous antigen, fragments thereof, or variants having the same functional activity.

As used herein, “foreign antigen” refers to a protein or fragment thereof, which is foreign to the recipient animal cell or tissue including, but not limited to, a viral protein, a parasite protein, an immunoregulatory agent, or a therapeutic agent.

The term “endogenous antigen” is used herein to refer to a protein or part thereof that is naturally present in the recipient animal cell or tissue, such as a cellular protein, an immunoregulatory agent, or a therapeutic agent.

The foreign antigen may be a protein, an antigenic fragment or antigenic fragments thereof that originate from viral and parasitic pathogens.

Alternatively, the foreign antigen may be encoded by a synthetic gene and may be constructed using conventional recombinant DNA methods; the synthetic gene may express antigens or parts thereof that originate from viral and parasitic pathogens. These pathogens can be infectious in humans, domestic animals or wild animal hosts.

The foreign antigen can be any molecule that is expressed by any viral or parasitic pathogen prior to or during entry into, colonization of, or replication in their animal host.

The viral pathogens, from which the viral antigens are derived include, but are not limited to, Orthomyxoviruses, such as influenza virus (Taxonomy ID: 59771); Retroviruses, such as RSV, HTLV-1 (Taxonomy ID: 39015) and HTLV-II (Taxonomy ID: 11909); Herpes viruses, such as EBV (Taxonomy ID: 10295), CMV (Taxonomy ID: 10358) or herpes simplex virus (ATCC #: VR-1487); Lentiviruses, such as HIV-1 (Taxonomy ID: 12721) and HIV-2 Taxonomy ID: 11709); Rhabdoviruses, such as rabies; Picornoviruses, such as Poliovirus (Taxonomy ID: 12080); Poxviruses, such as vaccinia Taxonomy ID: 10245); Rotavirus Taxonomy ID: 10912); and Parvoviruses, such as adeno-associated virus 1 (Taxonomy ID: 85106).

Examples of viral antigens include, but are not limited to, the human immunodeficiency virus antigens Nef (National Institute of Allergy and Infectious Disease HIV Repository Cat. # 183; GenBank accession # AF238278), Gag, Env (National Institute of Allergy and Infectious Disease HIV Repository Cat. # 2433; GenBank accession # U39362), Tat (National Institute of Allergy and Infectious Disease HIV Repository Cat. # 827; GenBank accession # M13137), Rev (National Institute of Allergy and Infectious Disease HIV Repository Cat. # 2088; GenBank accession # L14572), Pol (National Institute of Allergy and Infectious Disease HIV Repository Cat. # 238; GenBank accession # AJ237568) and T cell and B cell epitopes of gp120 (Hanke and McMichael, AIDS Immunol Lett., 66:177 (1999); Hanke, et al., Vaccine, 17:589 (1999); Palker, et al., J. Immunol., 142:3612-3619 (1989)); the hepatitis B surface antigen (GenBank accession # AF043578); rotavirus antigens, such as VP4 (GenBank accession # AJ293721) and VP7 (GenBank accession # AY003871); influenza virus antigens, such as hemagglutinin (GenBank accession # AJ404627); nucleoprotein (GenBank accession # AJ289872); and herpes simplex virus antigens, such as thymidine kinase (GenBank accession # AB047378).

The bacterial pathogens, from which the bacterial antigens are derived, include but are not limited to, Mycobacterium spp., Helicobacter pylori, Salmonella spp., Shigella spp., E. coli, Rickettsia spp., Listeria spp., Legionella pneumoniae, Pseudomonas spp., Vibrio spp., and Borellia burgdorferi.

Examples of protective antigens of bacterial pathogens include the somatic antigens of enterotoxigenic E. coli, such as the CFA/I fimbrial antigen and the nontoxic B-subunit of the heat-labile toxin; pertactin of Bordetella pertussis, adenylate cyclase-hemolysin of B. pertussis, fragment C of tetanus toxin of Clostridium tetani, OspA of Borellia burgdorferi, protective paracrystalline-surface-layer proteins of Rickettsia prowazekii and Rickettsia typhi, the listeriolysin (also known as “Llo” and “Hly”) and/or the superoxide dismutase (also know as “SOD” and “p60”) of Listeria monocytogenes; the urease of Helicobacter pylori, and the receptor-binding domain of lethal toxin and/or the protective antigen of Bacillus anthrax.

Example of antigens from biological weapons or pathogens include, but are not limited to, smallpox, anthrax, tularemia, plague, listeria, brucellosis, hepatitis, vaccinia, mycobacteria, coxsackievirus, tuberculosis, malaria, erhlichosis and bacterial meningitis.

The parasitic pathogens, from which the parasitic antigens are derived, include but are not limited to, Plasmodium spp., such as Plasmodium falciparum (ATCC#: 30145); Trypanosome spp., such as Trypanosoma cruzi (ATCC#: 50797); Giardia spp., such as Giardia intestinalis (ATCC#: 30888D); Boophilus spp.; Babesia spp., such as Babesia microti (ATCC#: 30221); Entamoeba spp., such as Entamoeba histolytica (ATCC#: 30015); Eimeria spp., such as Eimeria maxima (ATCC# 40357); Leishmania spp., (Taxonomy ID: 38568); Schistosome spp., such as Schistosoma mansoni (GenBank accession # AZ301495); Brugia spp., such as Brugia malayi (GenBank accession # BE352806); Fascida spp., such as Fasciola hepatica (GenBank accession # AF286903); Dirofilaria spp., such as Dirofilaria immitis (GenBank accession # AF008300); Wuchereria spp., such as Wuchereria bancrofti (GenBank accession # AF250996); and Onchocerea spp; such as Onchocerca volvulus (GenBank accession # BE588251).

Examples of parasite antigens include, but are not limited to, the pre-erythrocytic stage antigens of Plasmodium spp. such as the circumsporozoite antigen of P. falciparum (GenBank accession # M22982) P vivax (GenBank accession # M20670); the liver stage antigens of Plasmodium spp, such as the liver stage antigen 1 (as referred to as LSA-1; GenBank accession # AF086802); the merozoite stage antigens of Plasmodium spp; such as the merozoite surface antigen-1 (also referred to as MSA-1 or MSP-1; GenBank accession # AF199410); the surface antigens of Entamoeba histolytica, such as the galactose specific lectin (GenBank accession # M59850) or the serine rich Entamoeba histolytica protein (also referred to as SREHP; Zhang and Stanley, Vaccine, 18:868 (1999)); the surface proteins of Leishmania spp, such as 63 kDa glycoprotein (gp63) of Leishmania major (GenBank accession # Y00647 or the 46 kDa glycoprotein (gp46) of Leishmania major; paramyosin of Brugia malayi (GenBank accession # U77590; the triose-phosphate isomerase of Schistosoma mansoni (GenBank accession # W06781; the secreted globin-like protein of Trichostrongylus colubriformis (GenBank accession # M63263; the glutathione-S-transferases of Fasciola hepatica (GenBank accession # M77682; Schistosoma bovis (GenBank accession # M77682); S. japonicum (GenBank accession # U58012; and KLH of Schistosoma bovis and S. japonicum (Bashir, et al., supra).

Examples of tumor specific antigens include prostate specific antigen (PSA), TAG-72 and CEA; human tyrosinase (GenBank accession # M27160); tyrosinase-related protein (also referred to as TRP; GenBank accession # AJ132933); and tumor-specific peptide antigens.

Examples of transplant antigens include the CD3 molecule on T cells and histocompatibility antigens such as HLA A, HLA B, HLA C, HLA DR and HLA.

Examples of autoimmune antigens include IAS β chain, which is useful in therapeutic vaccines against autoimmune encephalomyelitis (GenBank accession # D88762); glatamic acid decarboxylase, which is useful in therapeutic vaccines against insulin-dependent type 1 diabetes (GenBank accession # NM013445); thyrotropin receptor (TSHr), which is useful in therapeutic vaccines against Grave's disease (GenBank accession # NM000369) and tyrosinase-related protein 1, which is useful in therapeutic vaccines against vitiligo (GenBank accession # NM000550).

Immunoglobulins

In the present invention, any soluble immunoglobulin or fragment thereof may be used including IgA, IgD, IgE, IgG, and IgM. Soluble antibodies are found in the blood and tissue fluids, as well as many secretions. In structure, they are globulins (in the γ-region of protein electrophoresis). They are synthesized and secreted by plasma cells that are derived from the B cells of the immune system. The five types of immunoglobulins are classified according to differences in their heavy chain constant domains and differs in its biological properties. The basic unit of each antibody is a monomer (one Ig unit) which is a “Y”-shaped molecule that consists of four polypeptide chains; two identical heavy chains and two identical light chains connected by disulfide bonds.

Preferrably, the imunnoglobulin is IgG or a fragment thereof wherein there are four subclasses including IgG1 (66%), IgG2 (23%), IgG3 (7%) and IgG4 (4%). More preferably, one of the heavy chains of IgG, and still more preferably, one of the heavy chains of IgG1 is used in the antigen/Ig Ab fusion peptide.

Fusion of Antigen with Immunoglobulins

Methods of making fusion proteins, either recombinantly or by covalently linking two protein segments, are well known. Preferably, fusion proteins are expressed recombinantly, as products of expression constructs. Expression constructs of the invention comprise a polynucleotide which encodes one or more fusion proteins of the present invention.

The expression construct may be included in an expression vector wherein the polynucleotides are operatively linked to an enhancer-promoter including a prokaryotic or eukaryotic promoter.

A promoter is a region of a DNA molecule typically within about 100 nucleotide pairs in front of (upstream of) the point at which transcription begins (i.e., a transcription start site). T hat region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

Preferably, expression vectors of the present invention comprise polynucleotides that encode the fusion polypeptides of the present invention. Alternatively, such vectors or fragments can code larger polypeptides or peptides which nevertheless include the basic coding region. In any event, it should be appreciated that due to codon redundancy as well as biological functional equivalence, this aspect of the invention is not limited to a specific DNA sequence but instead any sequence that encodes the fusion polypeptide.

An expression vector of the present invention is useful both as a means for preparing quantities of the fusion polypeptides-encoding DNA itself, and as a means for preparing the encoded peptides. It is contemplated that where the fusion polypeptides of the invention are made by recombinant means, one can employ either prokaryotic or eukaryotic expression vectors as shuttle systems. Such a system is described herein which allows the use of bacterial host cells as well as eukaryotic host cells.

Where expression of recombinant polypeptide of the present invention is desired and a eukaryotic host is contemplated, it is also envisioned to employ a vector, such as a plasmid, that incorporates a eukaryotic origin of replication. Additionally, for the purposes of expression in eukaryotic systems, one desires to position the fusion polypeptide encoding sequence adjacent to and under the control of an effective eukaryotic promoter such as promoters used in combination with Chinese hamster ovary cells. To bring a coding sequence under control of a promoter, whether it is eukaryotic or prokaryotic, what is generally needed is to position the 5′ end of the translation initiation side of the proper translational reading frame of the polypeptide between about 1 and about 50 nucleotides 3′ of or downstream with respect to the promoter chosen. Furthermore, where eukaryotic expression is anticipated, one would typically desire to incorporate into the transcriptional unit which includes the fusion polypeptide, an appropriate polyadenylation site.

The pRc/CMV vector (available from Invitrogen) is an exemplary vector for expressing a fusion polypeptide in mammalian cells, particularly COS and CHO cells. A polypeptide of the present invention under the control of a CMV promoter can be efficiently expressed in mammalian cells. The pCMV plasmids are a series of mammalian expression vectors of particular utility in the present invention. The vectors are designed for use in essentially all cultured cells and work extremely well in SV40-transformed simian COS cell lines. The pCMV1, 2, 3, and 5 vectors differ from each other in certain unique restriction sites in the polylinker region of each plasmid. The pCMV4 vector differs from these 4 plasmids in containing a translation enhancer in the sequence prior to the polylinker. While they are not directly derived from the pCMV1-5 series of vectors, the functionally similar pCMV6b and c vectors are available from the Chiron Corp. of Emeryville, Calif. and are identical except for the orientation of the polylinker region which is reversed in one relative to the other.

### Transfected Cells

In yet another embodiment, the present invention provides recombinant host cells transformed or transfected with a polynucleotide that encodes a fusion polypeptide of the present invention, as well as transgenic cells derived from those transformed or transfected cells. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection.

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

A transfected cell can be prokaryotic or eukaryotic. Preferably, the host cells of the invention are eukaryotic host cells such as COS cells.

In another aspect, the recombinant host cells of the present invention are prokaryotic host cells, including bacterial cells of the DH5a strain of Escherichia coli. In general, prokaryotes are preferred for the initial cloning of DNA sequences and constructing the vectors useful in the invention. For example, E. coli K12 strains can be particularly useful. Other microbial strains which can be used include E. coli B, and E. coli X1776 (ATCC No. 31537). These examples are, of course, intended to be illustrative rather than limiting.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli can be transformed using pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own polypeptides.

Those promoters most commonly used in recombinant DNA construction include the .beta.-lactamase (penicillinase) and lactose promoter systems and a tryptophan (TRP) promoter system. While these are the most commonly used, other microbial promoters have been discovered and utilized, and details concerning their nucleotide sequences have been published, enabling a skilled worker to introduce functional promoters into plasmid vectors.

For use in mammalian cells, the control functions on the expression vectors are often derived from viral material. For example, commonly used promoters are derived from polyoma, Adenovirus 2, Cytomegalovirus and most frequently Simian Virus 40 (SV40). The early and late promoters of SV40 virus are particularly useful because both are obtained easily from the virus as a fragment which also contains the SV40 viral origin of replication. Smaller or larger SV40 fragments can also be used, provided there is included the approximately 250 by sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

### Transformed Cells

In yet another embodiment, the present invention contemplates a process of preparing a fusion polypeptide by transfecting cells with a polynucleotide that encodes a fusion polypeptide of the present invention to produce transformed host cells; and maintaining the transformed host cells under biological conditions sufficient for expression of the polypeptide. Preferably, the transformed host cells are eukaryotic cells. Alternatively, the host cells are prokaryotic cells.

A host cell used in the process is capable of expressing a functional, recombinant fusion polypeptide. A preferred host cell is a Chinese hamster ovary cell. However, a variety of cells are amenable to a process of the invention, for instance, yeasts cells, human cell lines, and other eukaryotic cell lines known well to those of the art.

Following transfection, the cell is maintained under culture conditions for a period of time sufficient for expression of a fusion polypeptide of the present invention. Culture conditions are well known in the art and include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C. pH is preferably from about a value of 6.0 to a value of about 8.0, more preferably from about a value of about 6.8 to a value of about 7.8 and, most preferably about 7.4. Other biological conditions needed for transfection and expression of an encoded protein are well known in the art.

Transfected cells are maintained for a period of time sufficient for expression of a fusion polypeptide of the present invention. A suitable time depends inter alia upon the cell type used and is readily determinable by a skilled artisan. Typically, maintenance time is from about 2 to about 14 days.

A recombinant fusion polypeptide is recovered or collected either from the transfected cells or the medium in which those cells are cultured. Recovery comprises isolating and purifying the recombinant polypeptide. Isolation and purification techniques for polypeptides are well known in the art and include such procedures as precipitation, filtration, chromatography, electrophoresis and the like.

In use, the antigen-IgG peptide specifically binds to the antigen-specific B cells and labels them with the antigen-IgG peptide. The antigen-IgG peptide is preferably conjugated to a reporter group, such as a radiolabel (e.g., ³² P) or fluorescent label, an enzyme, a substrate, a solid matrix, or a carrier (e.g., biotin or avidin) to facilitate detection of specific levels of molecules or the specific binding activity of particular molecules of the present invention. Further, the antigenic/IgG peptide can be in solution or can be affixed to a solid substrate, such as a glass or plastic slide or tissue culture plate or latex, polyvinylchloride, or polystyrene beads.

Antigen-specific B cells which are bound to the antigen-IgG fusion peptide can be separated from cells which are not bound. Any method known in the art can be used to achieve this separation, including plasmapheresis, flow cytometry, or differential centrifugation.

Antigen-specific B cells which have been isolated from a patient can be treated with a reagent, such as a cytokine, a chemotherapeutic agent, or an antibody, and reinfused into the patient to provide a therapeutic effect. Optionally, the number of antigen-specific B cells which are bound to the antigen-IgG peptide can be quantified or counted, for example by flow cytometry, magnet beads, such as Stemcell or Dynamax, or any system that will provide for isolation and identification of the antigen-specific B cells.

In addition, the method and system of the present invention can be used to generate human monoclonal antibodies on demand. This can be accomplished by immortalization of the antigen-specific B cells with Epstein Bar Virus (EBV) or by conventional hybridoma methods using a human fusion partner. Both of these methods have been used to develop human monoclonal antibodies but they are inefficient and require that the B cell donor be immune to the test antigen. Immunization in vitro prior to B cell immortalization via EBV or cell fusion obviates the need to identify immune cell donors or to deliberately immunize a cell donor with the test vaccine. Thus, the method and system of the present invention circumvents the problem of having to identify immune individuals as a source of B cells to generate monoclonal antibodies. This is particularly important in situations when therapeutic antibodies are desired where it is difficult (or not possible) to deliberately immunize an individual and the target disease is rare.

The isolated antigen-specific B cells generated by the system of the present invention are suitable for fusion with a myeloma line for the ultimate production of monoclonal antibodies. Specialized myeloma cell lines have been developed from lymphocyte tumors for use in hybridoma-producing fusion procedures [G. Kohler and C. Milstein, Europe. J. Immunol. 6: 511-519)1976); M. Shulman et al., Nature 276: 269-270 (1978)]. It is preferred that human myeloma cells are used in the fusion procedure. The myeloma cells are introduced into the system with the inclusion of an agent that promotes the formation of the fused myeloma and B-cells, such as polyethylene glycol (PEG) and Dimethyl sulfoxide (DMSO).

Methods for generating hybrids of antibody-producing B-cells and myeloma cells usually comprise mixing B cells with myeloma cells in a 2:1 proportion (though the proportion may vary from about 20:1 to about 1:1), respectively, in the presence of an agent or agents that promote the fusion of cell membranes. Fusion procedures usually produce viable hybrids at very low frequency and as such, it is essential to have a means of selecting the fused cell hybrids from the remaining unfused cells, particularly the unfused myeloma cells. The antigen/IgG peptides of the present invention provide a means of detecting the desired antibody-producing hybridomas among other resulting fused cell hybrids.

Generally, the selection of fused cell hybrids is accomplished by culturing the cells in media that support the growth of hybridomas but prevent the growth of the myeloma cells which normally would go on dividing indefinitely. (The B-cells used in the fusion do not maintain viability in in vitro culture and hence do not pose a problem.) Generally, the myeloma cells used in the fusion lack hypoxanthine phosphoribosyl transferase. These cells are selected against in hypoxanthine/aminopterin/thymidine (HAT) medium, a medium in which the fused cell hybrids survive due to the HPRT-positive genotype of the spleen cells. The use of myeloma cells with different genetic deficiencies (e.g., other enzyme deficiencies, drug sensitivities, etc.) that can be selected against in media supporting the growth of genotypically competent hybrids is also possible.

Several weeks are required to selectively culture the fused cell hybrids. Early in this time period, it is necessary to identify those hybrids which produce the desired antibody so that they may be subsequently cloned and propagated. The antigen/IgG1 peptides of the present invention can be used to identify such hybrids.

Once the desired fused cell hybrids have been selected and cloned into individual antibody-producing cell lines, each cell line may be propagated in vitro in laboratory culture vessels; the culture medium, also containing high concentrations of a single specific monoclonal antibody, can be harvested by decantation, filtration or centrifugation

In the alternative, antibody producing B-cells can be suspended in EBV infected culture supernatant and incubated. The EBV infected B-cells are immortalizes upon infection. Notably the EBV infected culture may be introduced at the same time as the naive B cell or subsequent to formation of antibody producing B cells. Also, these lymphocytes may be fused to an appropriate fusion partner in order to produce a stable, monoclonal producing hybridoma.

### Screening Assays for Antigen-Specific B Cells

The present invention provides a process of screening a biological sample for the presence of an Antigen-specific B cells. A biological sample to be screened can be a biological fluid such as extracellular or intracellular fluid or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide.

In accordance with a screening assay process, a biological sample suspected of including antigen-specific B cells is exposed to the antigen-IgG fusion peptide of the present invention. Typically, exposure is accomplished by forming an admixture in a liquid medium that contains both the antigen-specific B cell and the antigen-IgG fusion peptide of the present invention. Further the antigen-IgG fusion peptide of the present invention can be affixed to a solid support as long as the major epitope directed to the antigen-specific B cell has maintained the functional tertiary structure. The biological sample is exposed under biological reaction conditions and for a period of time sufficient for a complex to form between the antigen-IgG fusion peptide and the antigen-specific B cell. Biological reaction conditions include ionic composition and concentration, temperature, pH and the like. Ionic composition and concentration can range from that of distilled water to a 2 molal solution of NaCl. Temperature preferably is from about 25° C. to about 40° C. pH is preferably from about a value of 4.0 to a value of about 9.0, more preferably from about a value of 6.5 to a value of about 8.5 and, even more preferably from about a value of 7.0 to a value of about 7.5. The only limit on biological reaction conditions is that the conditions selected allow for the complex to form between the antigen-IgG fusion peptide and the antigen-specific B cell and that the conditions do not adversely affect either component.

Exposure time will vary inter alia with the biological conditions used, the concentration of antibody and peptide and the nature of the sample (e.g., fluid or tissue sample). Means for determining exposure time are well known to one of ordinary skill in the art. Typically, exposure time is from about 10 minutes to about 200 minutes.

The presence of antigen-specific B cells in the sample is detected by detecting the formation and presence of the antigen-IgG fusion peptide and the antigen-specific B cell complex. Means for detecting such complexes are well known in the art and include such procedures as flow cytometry, centrifugation, affinity chromatography or a binding of a secondary antibody to the formed complex.

In one embodiment, detection is accomplished by detecting an indicator affixed to the antibody. Exemplary and well known such indicators include radioactive labels (e.g., ³²P, ¹²⁵I, ¹⁴C), a second antibody or an enzyme such as horse radish peroxidase. Means for affixing indicators to antibodies are well known in the art. Commercial kits are available.

The antigen specific antibody producing B cells may also be generated in vitro by the method described in co-pending U.S. Provisional Application No. 60/747,021 filed in the United States Patent and Trademark Office on May 11, 2006 entitled “A GENERAL METHOD FOR GENERATING HUMAN ANTIBODY RESPONSES IN VITRO” the contents of which are incorporated by reference herein for all purposes.

In the practice the method of producing in vitro antigen specific antibody producing B cells may be accomplished by drawing blood from a donor and monocytes, naive T cells and naive B cells are separated therefrom. The human whole blood can be collected in heparin containing tubes. Although this is the preferred method of obtaining whole blood, any other method, such as using a needle and heparin, ACD, Citrate or EDTA coated syringe, is acceptable. The monocytes and peripheral blood lymphocytes can be separated using a density gradient such as Ficoll-Hypaque™ (Pharmacia Biotechnology Group, Uppsala, Sweden). Other methods, including magnetic bead assisted separation (MACs and Dynel technologies, that are capable of separating the desired components from the rest of the components of the whole blood are also acceptable.

Once the monocytes are separated from the PBL, the monocytes are matured into monocytes derived dendritic cells with any agent that promotes monocyte maturation. The monocytes are differentiated into monocyte derived dendritic cells (MDCs) by culturing for 4 to 7 days in the presence of activation agents. For example, stem-cell-derived- or monocyte-derived DCs can be sustained ex vivo with GM-CSF and other cytokines and can be matured in vitro by bacteria, viruses, fungi, bacterial products, such as lipopolysaccharide (LPS), inflammatory stimuli, and cytokines, including interferons, interleukin-1 (IL-1), tumor necrosis factor alpha (TNF-α) and its superfamily, RANTES, and most often, by CD40 ligand, which plays an important role in DC/T-cell interaction. Specifically, the monocyte maturation-promoting agent may be any compound which facilitates the development and differentiation of monocytes to dendritic cells. Suitable monocyte maturation-promoting agents include, but not limited to the following agents IL-1, GM-CSF, IL-3, IL-4, IL-6, TNF-α, G-CSF, M-CSF, IL-12, IL-15, IL-18 or mixture thereof. Preferably, the monocyte maturation-promoting agent is GM-CSF alone or in combination with an additional maturation agent. Further any additional component that increases the rate of monocyte maturation may also be included, such as histamine. Optimal conditions for culturing should be considered including temperature, humidity, pH and the addition of carbon dioxide with a timeframe ranging from about 4 to 10 days.

Maturation stimulates increased expression of HLA-DR, CD40, and costimulatory molecules and secretion of cytokines which is important because MDCs are the sole population of antigen presenting cells in vivo that initiate primary immune responses. Notably, it should be recognized that yields and types of expressed receptors can be influenced by culture components. For example, the culture medium can be serum free or include autologous serum and plasma with a differential effect observed in the phenotypic characterization of these culture-derived DC.

Once the monocytes have matured into monocyte dendritic cells, they are isolated from the culture medium and combined with naive T-cells and naïve B-cells, all cells preferably from a single donor, and a target antigen for production of antibodies specifically for the target antigen.

Additionally an adjuvant or combination of adjuvants may be included in the culture medium to enhance the immune response including, but not limited to, the A subunit of cholera toxin or parts thereof (e.g. the A1 domain of the A subunit of Ctx from any classical Vibrio cholerae or El Tor V. cholerae strain. Alternatively, any bacterial toxin that increases cellular cAMP levels, such as a member of the family of bacterial adenosine diphosphate-ribosylating exotoxins may be used in place of CtxA, for example the A subunit of heat-labile toxin (referred to herein as EltA) of enterotoxigenic Escherichia coli, pertussis toxin S1 subunit; as a further alternative the adjuvant may be one of the adenylate cyclase-hemolysins of Bordetella pertussis, Bordetella bronchiseptica or Bordetella parapertussis, B. parapertussis or B. bronchiseptica.

Other adjuvants that may be used in the present invention include cytokines, such as IL-4, IL-5, IL-6, IL-10, 11-12, 11-18, TGFβ or M60316, IFN-Y and TNFα or chemokines, such as MIP-1α, MIP-1β, MIP3α, MDC, RANTES, IL-8, and SDF-1α. Notably, the adjuvant may be chosen to provide for not only the production of primary antibodies IgM but also other types such as IgG and IgA.

The antigen specific antibody producing B cells generated by this method can be separated by the method described herein.

The following examples describe the new inventive method. These examples are given merely for illustration of the present invention and are not to be construed as a limitation on the remainder of the specification in any way.

### Example 1

The method employs a chimeric protein antigen made by fusing the antigenic sequence with the heavy chain of a human IgG1 protein and detecting the binding of the antigen-IgG1 fusion peptide to B cells by flow cytometry. This is illustrated by using a single-chain gp120-CD4 complex (FLSC (1)) as the example antigen. FLSC is comprised of the outer membrane envelope glycoprotein, gp120, of HIV-1 and the D1 D2 domains of its canonical receptor, CD4, linked by a flexible polypeptide spacer. This complex binds specifically to the CCR5 co-receptor found on CD4+ T cells and exposes epitopes associated with this process that are targets of antibodies that either neutralize HIV-1 or mediate antibody-dependent cell mediated cytotoxicity (ADCC) to HIV-1. These structures on FLSC are sensitive to chemical modification by agents that modify either amino or carboxyl groups making this molecule difficult to render fluorescent. The IgG1 chimeric protein was prepared by fusing the sequence for FLSC with that of the human IgG1 heavy chain (2). Generally, 293 cells were transiently transfected with the plasmid containing nucleotides sequences encoding the chimeric polypeptide gp120-CD4-IgG1 and the expressed protein was characterized by immunoblotting of the culture supernatants.

FLSC-IgG1 is secreted as a disulfide bonded dimer and can be purified from culture supernatants by protein-A affinity columns. The FLSC-IgG1 fusion peptide was rendered fluorescent by the simple admixture of a fluorescently tagged Fab fragment specific for the Fc region of human IgG1 followed by quenching with an excess of non-specific human IgG. This was done using a commercially available kit from Invitrogen (Zenon® binding reagent) but it could also be done using laboratory generated reagents or by fusing the IgG1 CH3 region with a fluorescent reporter molecule. None of these modifications affect the structure of the FLSC moiety.

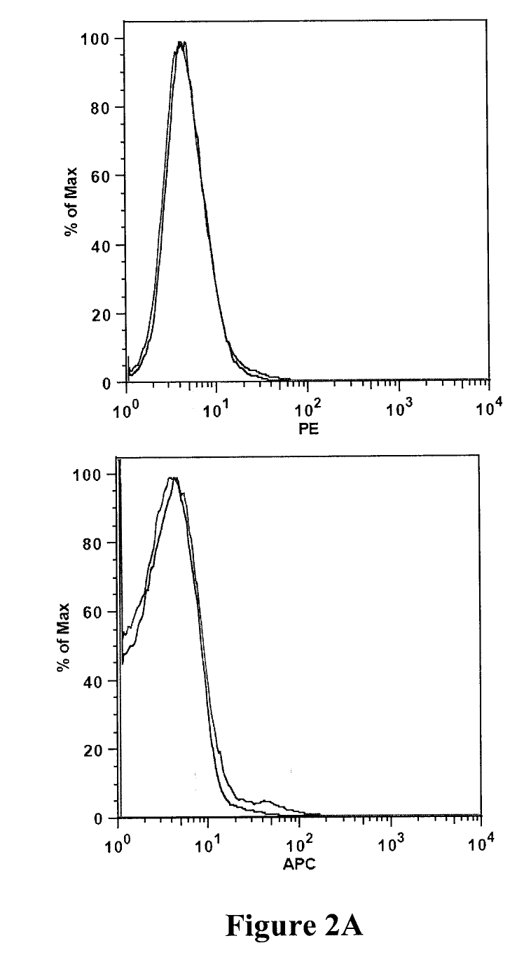

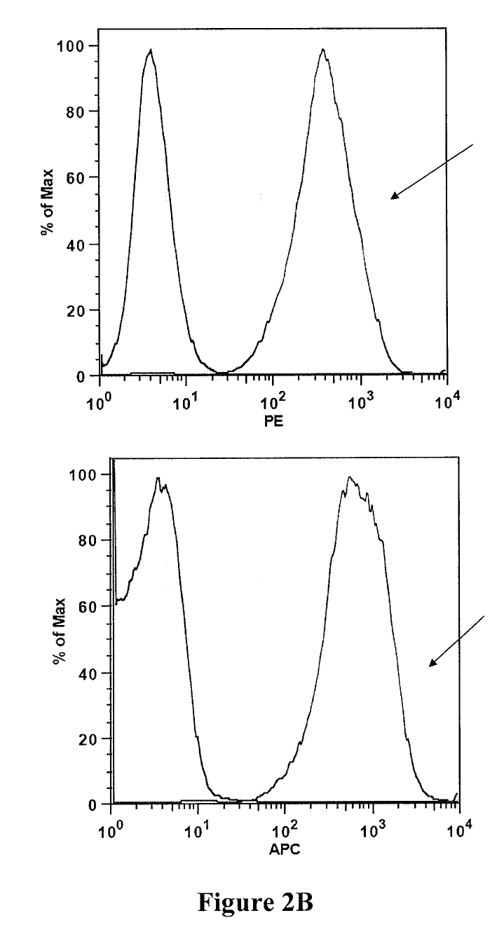

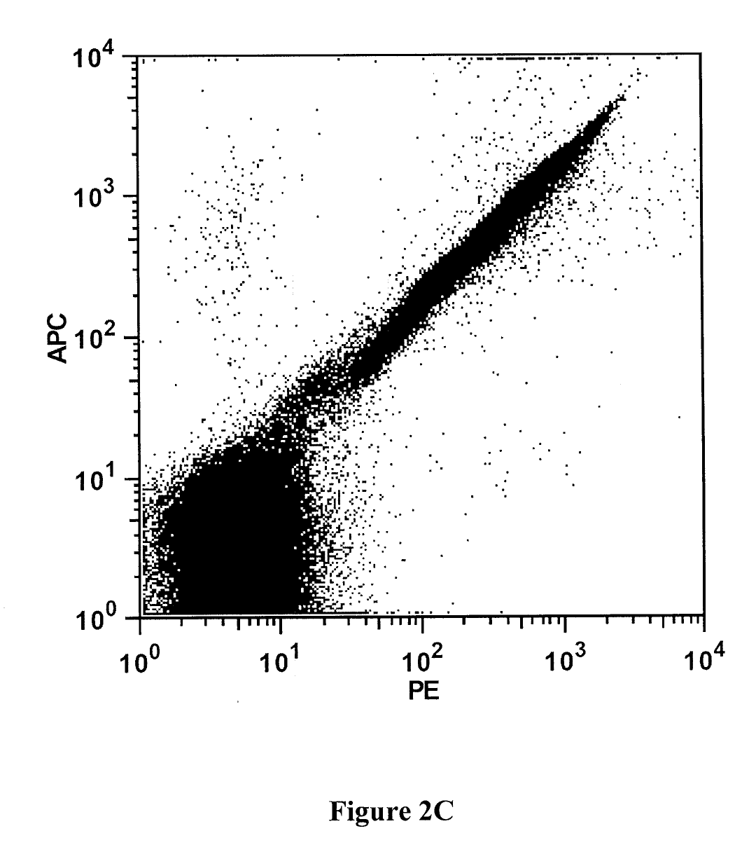

As a first test to determine whether the fluorescent FLSC-IgG1 retains the function of unlabeled FLSC, a saturating concentration of PE, phycoerythrin or APC, allophycocyanin labeled FLSC-IgG1 was reacted with transfected dog lymphocytes that express CCR5 or their non-transfected parents followed by washing, fixing with paraformaldehyde, and analysis by flow cytometry. The results of this experiment are shown in FIG. 2. As can be seen by the results set forth in FIG. 2, there was no binding of either PE or APC FLSC-IgG1s to the parental cell line Cf2Th (FIG. 2A) whereas both bound specifically to the CCR5+ derivative, Cf2Th CCR5 (arrow shown in FIG. 2B). In the flow histogram, the lines are the cells exposed to the fluorescent Fab reagent alone and the lines with arrows are that reagent plus the FLSC-IgG1 complex. FIG. 2C is a bivariate flow histogram for the binding of equimolar concentrations of the fluorescent FLSC-IgG1 complexes to either the parental cells (light gray) or the CCR5+cells (dark gray). As expected there is a 1:1 correspondence of binding for the two labeled FLSC-IgG1 molecules to the CCR5+ cells indicating that the two different labeling conditions produce products of equivalent reactivity. The use of two independent labels for the same antigen greatly increases the ability to detect rare antigen binding B cells (3) by flow cytometry. In addition, the ability to use multiple labels makes it possible to use different antigen-IgG1 chimeras that have related but distinct epitopes to carry out detailed analyses of antibody specificity at the single B cell level. This is potentially very useful in clinical settings or in vaccine trials where it is key to know the frequencies of B cells for different epitopes on the same antigen.

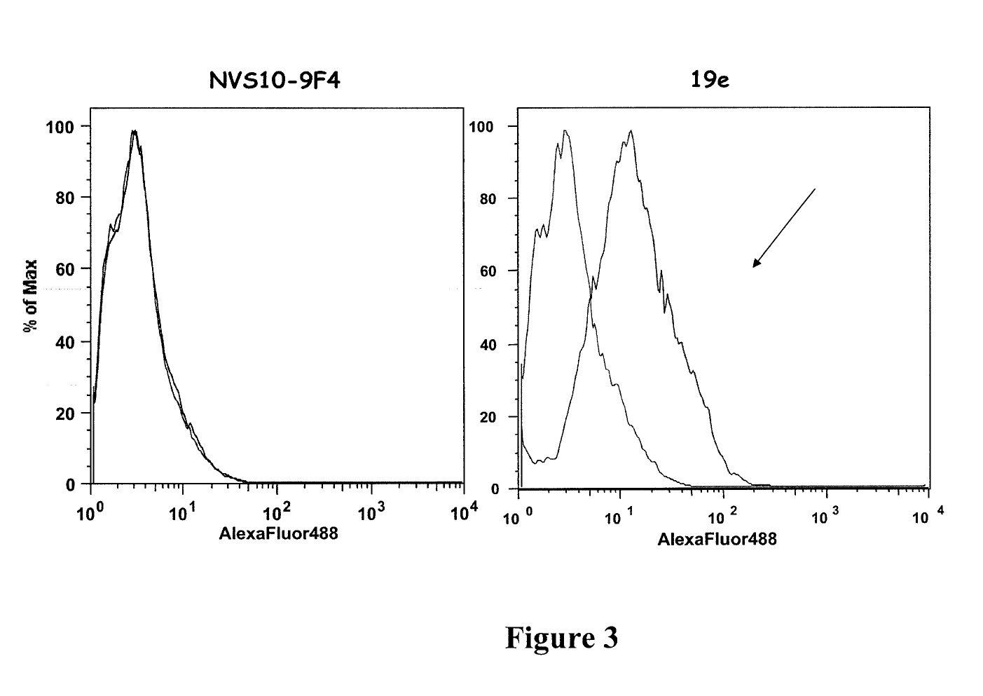

The study described above used non-B cells transfected with a receptor for FLSC as a first step toward validating this method. Additionally, a model B cell clone was used to verify that fluorescent FLSC-IgG1 can bind to antigen-specific B cells. This clone, designated 19e, produces an antibody that specifically recognizes a complex epitope made when gp120 binds to CD4 in the FLSC moiety. This epitope is not expressed on free gp120 or CD4. Clone 19e is an Epstein Barr Virus transformed cell line that was originally isolated from an HIV-1 infected individual and it retains the surface IgG that functions as an antigen receptor. As such it is an excellent model for human memory B cells. The results of a FLSC-IgG1 binding study using a control EBV B cell line NVS10-9F4 or 19e as the target cells are shown in FIG. 3 for FLSC-IgG1 rendered fluorescent with Fab anti-IgG1Fc labeled with AlexaFluor-488D.

In this study, it is evident that the cells exposed to reagent plus FLSC-IgG1 (arrow) showed binding for the 19e cells but not for the negative control NVS10-9F4 cells.

Taken together, the above studies show that an antigen-IgG1 chimeric molecule can be rendered fluorescent in a simple and non-destructive fashion such that it retains its native biological and immunochemical conformations. This provides proof of concept data that this approach works and applicable for any protein that can successfully be fused with an IgG1 heavy chain constant region as was done for FLSC thus providing a general way to make native fluorescent reagents for receptor binding studies. In the above example both CCR5 binding and anti-FLSC IgG binding were demonstrated for the FLSC-IgG1 molecule. These results show that both CCR5+ T cells and B cells that bear surface immunoglobulin molecules that recognize epitopes on FLSC can be identified and potentially purified by a variety of methods including flow sorting. This method should be particularly useful for the identification and purification of antigen-specific B cells as a first step in monoclonal antibody production. This would greatly increase the frequencies of positive clones at an early step either in direct PCR cloning of variable regions genes or in generating antigen-specific cell lines by immunization in vitro. In addition, it should allow for the direct quantification of B cells specific for individual epitopes on antigens of commercial or clinical interest.

### REFERENCES

The contents of all references cited herein are hereby incorporated by reference herein for all purposes.
1. Fouts, T. R., R. Tuskan, K. Godfrey, M. Reitz, D. Hone, G. K. Lewis, and A. L. DeVico. 2000. Expression and characterization of a single-chain polypeptide analogue of the human immunodeficiency virus type 1 gp120-CD4 receptor complex. J Virol 74:11427-11436.
2. Vu, J. R., T. Fouts, K. Bobb, J. Burns, B. McDermott, D. I. Israel, K. Godfrey, and A. DeVico. 2006. An immunoglobulin fusion protein based on the gp120-CD4 receptor complex potently inhibits human immunodeficiency virus type 1 in vitro. AIDS Res Hum Retroviruses 22:477-490.
3. Townsend, S. E., C. C. Goodnow, and R. J. Cornall. 2001. Single epitope multiple staining to detect ultralow frequency B cells. J Immunol Methods 249:137-146.

That which is claimed is:

1. A method for isolating antigen-specific B-cells induced by exposure to a specific epitope of a target antigen of choice, the method comprising:
modifying the antigen of choice with the fusion of at least a fragment of an Ig Ab or variant thereof to generate an antigen-IgAb fusion peptide;
contacting a sample comprising B cells; and
detecting the binding of the antigen-Ig Ab fusion peptide to antigen-specific B cells.

2. The method according to claim 1, wherein the IgAb is the heavy chain of the human IgG1 protein.

3. The method according to claim 1, further comprising a fluorescent tag, wherein the fluorescent tag is a fluorescently tagged Fab fragment specific for the Fc region of human IgAb.

4. The method according to claim 1, further comprising a fluorescent tag, wherein the fluorescent tag specific for a region of the human IgAb

5. The method according to claim 1, wherein the detecting the binding of the antigen-Ig Ab fusion peptide to antigen-specific B cells is detected by flow cytometry.

6. The method according to claim 1, wherein the antigen is a reactive epitope.

7. The method of claim 1, wherein multiple and different antigen-IgG1 chimeras are used to determine antibody specificity on the B cell.

8. The method of claim 1, wherein the antigen is a gp120/cd4 chimeric.

9. The method according to claim 1, wherein the IgAb is IgG1.

10. The method according to claim 5, wherein the antigen specific B-cells are quantified.

11. The method according to claim 10, wherein the quantification corresponds to the immune response in reaction to the antigen.

12. The method according to claim 1, wherein the a sample comprising B cells is a blood sample from a donor and at least naive T cells, naive B cells and monocytes are isolated from the sample.

13. The method according to claim 12, further comprising differentiating the monocytes into monocyte derived dendritic cells and culturing the monocytes derived dendritic cells with T cell, B cells and the target antigen in vitro to generate antigen-specific antibody producing B cells.

14. The method according to claim 13, further comprising contacting the antigen-Ig Ab fusion peptide to the antigen-specific antibody producing B cells and detecting the binding of the antigen-Ig Ab fusion peptide to antigen-specific antibody producing B cells.

15. A method of treatment comprising:
isolating B-cells from a subject:
modifying an antigen with the fusion of at least a fragment of an Ig Ab or variant thereof, wherein the antigen comprises a reactive epitope;
contacting the antigen-Ig Ab fusion peptide to the isolated B cells;
detecting the binding of the antigen-Ig Ab fusion peptide to antigen-specific B cells in a biological sample of a subject;
separating the antigen-specific B cells from the antigen-Ig Ab fusion peptide;
treating the antigen-specific B cells with a reagent including a cytokine, a chemotherapeutic agent, HIV therapeutic agent, chemokine or an antibody; and
reinfusing the treated antigen-specific B cells into the subject to provide a therapeutic effect.
